(19) United States
(12) Patent Application Publication
(10) Pub. No.: US 2020/0038242 A1
(43) Pub. Date: Feb. 6, 2020

(54) APPARATUS AND METHOD FOR PHACOEMULSIFICATION

(71) Applicant: NALLAKRISHNAN FAMILY TRUST, Willowbrook, IL (US)

(72) Inventor: Ravi Nallakrishnan, Westmont, IL (US)

(21) Appl. No.: 16/532,722

(22) Filed: Aug. 6, 2019

Related U.S. Application Data
(60) Provisional application No. 62/714,836, filed on Aug. 6, 2018.

(51) Int. Cl. A61F 9/007

(52) U.S. Cl. CPC A61F 9/00745; A61M 1/0058; A61M 2210/0612; A61F 9/00754

(57) ABSTRACT

A phacoemulsification needle is provided for emulsifying body tissue. The needle is adapted to be attached to a phacoemulsification handpiece that imparts a vibration to the needle. The needle has a hollow body with an operative distal end, a proximal end for being attached to a handpiece, and an internal surface defining an aspiration passage extending between the proximal and distal ends. The needle body includes a tip at the distal end and the needle body has a blockage reduction means located within the aspiration passage at a location proximal of the tip, wherein the blockage reduction means is one of: (i) a protrusion extending radially inward from the internal surface; (ii) a groove extending into the internal surface; (iii) at least one insert located within the aspiration passage; (iv) at least one blade located within the aspiration passage; or (v) a deformed portion of the needle body.

### FIELD OF THE INVENTION

This disclosure relates to surgical instruments used in ophthalmic surgery and methods of use thereof, and more particularly to phacoemulsification apparatuses and methods of use.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of about 12 mm in length.

Later techniques focused on removing diseased lenses and inserting replacement artificial lenses through as small an incision as possible, about 5 mm in length. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece attached to a proximal end of a hollow needle. In the handpiece, an electrical energy is applied to a piezoelectric crystal to vibrate the distal, working end of the needle at ultrasonic frequencies in order to fragment the diseased lens into small enough particles to be aspirated from the eye through an aspiration passage in the hollow needle. Commonly, an infusion sleeve is mounted around the needle at the distal end to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage may be permanent because these cells do not regenerate. Some benefits of using as small an incision as possible during such surgery are the minimization of leakage of liquid during and after surgery to help prevent tissue collapse, faster healing time, and decreased post-operative astigmatism.

Many phacoemulsification needles and tips are designed for use with handpieces that vibrate the needle longitudinally at relatively low frequencies. In addition to longitudinal vibration, certain handpieces impart a torsional motion to the needle at an oscillation frequency of about 100 cycles per second. There are also handpieces that provide torsional oscillation of the phacoemulsification tip at frequencies of about 32,000 cycles per second. Alternatively, some handpieces, such as the Cetus ARC Nano laser, utilize laser pulses with no moving mechanical parts to emulsify the nucleus of the eye.

Use of the torsional-type handpiece has called for phacoemulsification needle tip designs differing from those used with the longitudinal-type handpiece. For example, needles have been designed with tips that are shaped, swaged and angled to take advantage of the needle motion created by the handpiece.

There are known phacoemulsification systems, such as the Centurion® System manufactured by Alcon Laboratories of Ft. Worth, Tex., which allow the surgeon to choose between using torsional motion, longitudinal motion, or a blend thereof with a single handpiece. Other common systems include the Sovereign® System, Whitestar Signature® System, Signature Ellips® FX System manufactured by Johnson & Johnson of Santa Ana, Calif. and the Stellaris® System manufactured by Bausch & Lomb of Rochester, N.Y. Common frequencies for longitudinal oscillation range from 29 Hz to 43 Hz. Common frequencies for torsional oscillation range from 31 Hz to 38 Hz. A common blended setting uses torsional motion two-thirds of the time, and longitudinal motion one-third of the time. It is believed that the “blended” motion produces a more three-dimensional effect because of the back-and-forth motion imparted during longitudinal phacoemulsification and the eccentric motion produced at the tip during torsional phacoemulsification.

Many surgeons favor phacoemulsification needles having the straight tip design commonly used with longitudinal handpieces. The great majority of surgeons use longitudinal handpieces rather than the torsional handpieces, often because torsional phacoemulsification equipment is more expensive than longitudinal equipment, and thus these surgeons find themselves unable to take advantage of the enhanced phacoemulsification results claimed by the torsional phacoemulsification systems.

With reference to U.S. Pat. Nos. 8,764,782 and 8,992,459, which are incorporated by reference herein in their entireties, the inventor has previously found that forming a phacoemulsification needle having a tip in an off-axis position relative to the axis of the aspiration passage extending through the needle body causes an eccentric motion or “wobble” during torsional phacoemulsification and improves the efficiency of phacoemulsification. Surprisingly, the inventor has also found that forming the tip in such an off-axis position also increases the efficiency of phacoemulsification when using a longitudinal handpiece. Preliminary clinical examinations indicate that using an off-axis needle with a longitudinal handpiece may be more efficient than using the same needle with a torsional hand piece providing 100% torsional action, where efficiency is measured by the energy dissipated during phacoemulsification. When used herein, the term “dissipated energy” refers to the amount of energy, most commonly measured in joules, used by the handpiece during phacoemulsification. Lower dissipated energy readings mean that less heat is being produced during phacoemulsification, which in turn lowers the possibility of thermal damage to the delicate eye tissues.

Use of an off-axis tip with a longitudinal hand piece appears to create a hybrid type of phacoemulsification motion without using the more complex and expensive torsional phacoemulsification apparatus. The inventor has also determined that the eccentric or wobble type of motion can be imparted to a phacoemulsification needle with no flare at the tip by forming the central aspiration passage within the needle body in an off-axis position. It is also expected that similar results will be obtained using a straight phacoemulsification needle having an aspiration passage that is formed with a cross-sectional configuration different than the cross-sectional configuration of the needle body itself, and that these results will be further amplified if the passage is also placed off-axis.

The inventor has herein further determined that there is a need for further modification and improvement of phacoemulsification needles to provide beneficial fluid management to prevent or at least minimize collapse or flattening of the anterior chamber, without the need of purchasing an expensive fluidics management system.

The inventor has further found that some interior surfaces of a needle tip may result in unwanted bounce-back or ejection of tissue particles from the opening of the aspiration passage in the needle body instead of being aspirated through the aspiration passage and transported through the needle body. Such bounce-back, repulsion, or surge decreases the efficiency of the overall aspiration of the needle and may increase the time of surgery,

While the following describes a preferred embodiment or embodiments of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as described and claimed herein

### SUMMARY OF THE INVENTION

In accordance with one preferred embodiment of the present invention, a phacoemulsification needle is provided for emulsifying body tissue. The needle is adapted to be attached to a phacoemulsification handpiece imparting a vibration to the needle. The needle has a hollow body having a distal end, a proximal end, and an internal surface defining an aspiration passage extending between the proximal and distal ends. The distal end of the needle body is for mounting the needle body to a phacoemulsification handpiece. The needle body has a tip formed at its distal end. The aspiration passage defines a longitudinally-extending central body axis. The needle body has a blockage reduction means located within the aspiration passage at a location proximal of the tip, wherein the blockage reduction means is one of: (i) a protrusion extending radially inward from the internal surface; (ii) a groove extending into the internal surface; (iii) at least one insert located within the aspiration passage; (iv) at least one blade located within the aspiration passage; or (v) at least one indented portion of the needle body.

In accordance with another preferred embodiment of the present invention, a method of phacoemulsification is disclosed. The method includes the step of obtaining a phacoemulsification needle as described above. The method includes the step of assembling the proximal end of the phacoemulsification needle with a vibratory handpiece. The method includes the step of imparting a vibration to the needle with the handpiece to emulsify tissues in the eye. The method further includes the step of aspirating the emulsified tissues of the eye into the aspiration passageway to come into contact with the blockage reduction means.

It should be appreciated that the invention may include any of the detailed blockage reduction means described herein, either alone or in any combination. Furthermore, other objects, features and advantages of the invention will become apparent from a review of the entire specification including the appended claims and drawings.

### BRIEF DESCRIPTION OF THE DRAWINGS

FIG. 1 is an enlarged, side elevation view of a first embodiment of a phacoemulsification needle embodying the present invention;

FIG. 2 is a greatly enlarged fragmentary view of the tip portion of the needle illustrated in FIG. 1;

FIG. 3 is a greatly enlarged, front elevation view of the needle tip illustrated in FIG. 1;

FIG. 4 is a greatly enlarged cross-sectional view taken along plane 4-4 in FIG. 3 of the needle illustrated in FIG. 1;

FIG. 5 is a greatly enlarged, fragmentary perspective view of the body portion of the needle illustrated in FIG. 1, and FIG. 5 illustrates a plurality of raised internal protrusions extending in a helical fashion along the inside surface of the body portion of the needle;

FIG. 6 is an enlarged, fragmentary, side elevation view and a side elevation view of an alternative embodiment of the body portion of the needle illustrated in FIG. 1, and FIG. 6 illustrates a single helical internal groove extending within the inside surface of the body portion of the needle;

FIG. 7 is a series of views showing four additional alternative embodiments of the body portion of the needle illustrated in FIG. 1, and FIG. 7 shows indented portions of the needle body forming periodic raised internal surfaces or protrusions for assisting in preventing blockages of the aspiration passage;

FIG. 8 is a series of views of an additional alternative embodiment of the body portion of the needle illustrated in FIG. 1;

FIG. 9 is a greatly enlarged, fragmentary, perspective view of an alternative embodiment of the body portion of the needle illustrated in FIG. 1, and FIG. 9 illustrates a single helical insert extending within the aspiration passage of the body portion of the needle;

FIG. 10 is a greatly enlarged, fragmentary, perspective view of another alternative embodiment of the body portion of the needle illustrated in FIG. 1, and FIG. 10 illustrates a single helical insert extending within the aspiration passage of the body portion of the needle;

FIG. 11 is a series of enlarged views of another alternative embodiment of the body portion of the needle illustrated in FIG. 1, and FIG. 11 illustrates a pair of helical inserts extending within the aspiration passage of the body portion of the needle;

FIG. 12 is a greatly enlarged, partial, cross-sectional view of another alternative embodiment of the body portion of the needle illustrated in FIG. 1, and FIG. 12 illustrates a plurality of radial blades or fins extending within the aspiration passage of the body portion of the needle;

FIG. 13 is a greatly enlarged, cross-sectional view of another embodiment a phacoemulsification needle according to the present invention, and FIG. 13 illustrates a straight needle without any flaring or offset tip; and

FIG. 14 is a greatly enlarged end view of the needle illustrated in FIG. 13; and

FIG. 15 is a greatly enlarged, cross-sectional view of another embodiment a phacoemulsification needle according to the present invention, and FIG. 15 illustrates a needle with a flared, offset tip having a curved, sloping surface leading proximally to the aspiration passage.

### DETAILED DESCRIPTION OF THE PREFERRED EMBODIMENT

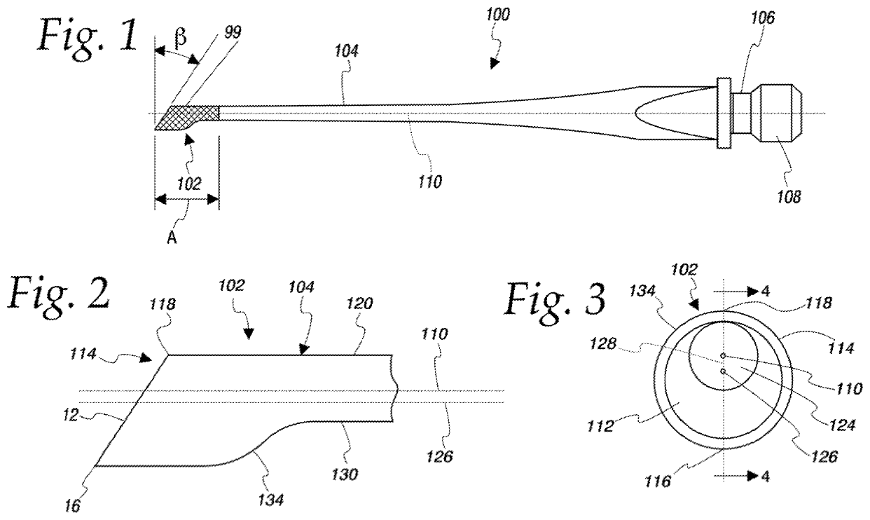

Referring now to FIG. 1, the numeral 100 indicates a first preferred embodiment of a phacoemulsification needle embodying the present invention. Needle 100 is generally straight and has a needle body 104. The body has an operative or distal end 99 and a proximal end 106, defining a length of the needle body 104. The needle distal end 99 has a tip 102. The tip 102 preferably has a leading and trailing edge defined by an angle beta that is about 30 degrees to the plane running perpendicular to the length of the needle body 104. The tip 102 need not be angled at all or may be defined by other angles. FIG. 2 further illustrates that the exterior surface “A” of the needle tip 102 is preferably textured or roughened.

Still referring to FIG. 1 the needle proximal end 106 may have a mounting portion or mating surfaces for connecting the needle 100 to a phacoemulsification handpiece (not shown). The needle 100 may be connected to the handpiece in any manner such as by mating threads, clamping, snap fit, lock, friction fit, or an adjustable fit. The needle body has an aspiration passage (124 in FIG. 2) running from the proximal end 106 to the distal end 99 and defining a central needle axis 110. As described hereinafter, directions inward or outward from the needle axis 110 are termed “radial” and directions along the axis 110 are either toward the distal end 99 or the proximal end 106.

Referring now to FIG. 2, which is a greatly enlarged view of the needle tip 102. The tip 102 can generally be characterized as having a flaring shape in which the aspiration passage 124 is radially widened at the needle body distal end 99 when compared to the radial height of the aspiration passage 124 in the remaining portion of the needle body 104 which is located proximally of the tip 102. The tip 102 may be characterized as having an open or hollow mouth 112 terminating in a lip 114. As previously discussed, the tip 102 may have a leading edge 116 and a trailing edge 118. The trailing edge 118 is preferably contiguous with the upper needle surface 120, while the leading edge 116 is laterally offset from the lower needle surface 130. However, in the broadest aspect of the invention, the tip 102 need not have any discernible leading or trailing edges, and the location of the leading and trailing edges may be positioned elsewhere along the lip 114.

Referring to FIG. 3, the tip 102 may have a central tip axis 126 that is offset from the needle body axis 110 by a distance 128. The aspiration passage 124 can be seen to connect to lip 114 via the open mouth 112. The first illustrated embodiment of the needle tip 102 can be seen to have a circular lip 114. Orientation of the tip axis 126 to be offset from the body axis 110 may provide beneficial eccentric motion to the phacoemulsification needle distal end 99 during vibratory oscillation (longitudinal, torsional, or a blend thereof) by a handpiece.

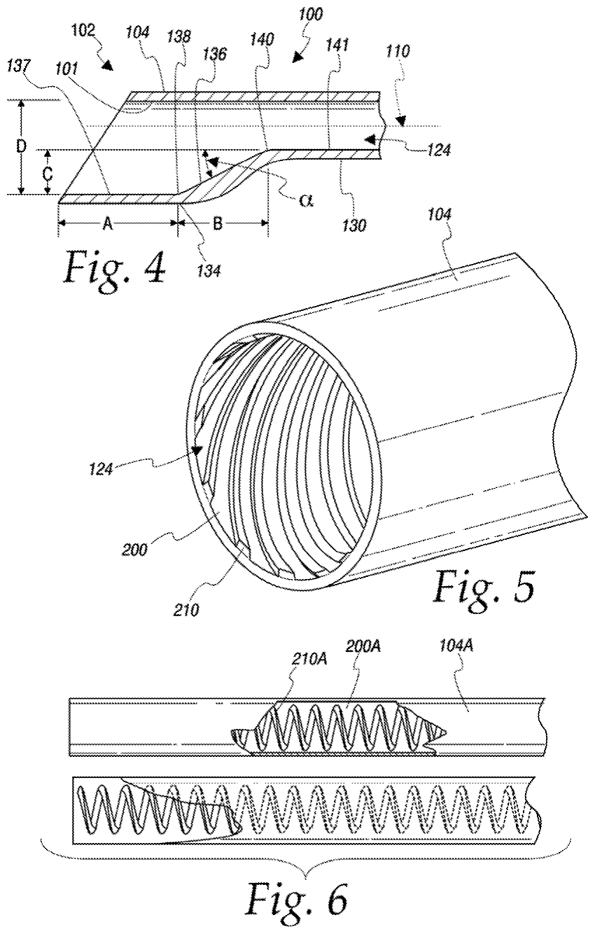

Referring next to FIG. 4, which is a cross-sectional view taken along plane 4-4 of FIG. 3, the interior features of the needle distal end 99 and the needle tip 102 can be seen in detail. The first illustrated embodiment of the needle 100 shows that the tip 102 has an upper surface 103 that is coextensive with the upper surface of the aspiration passage 124 in the needle body 104. An offset portion 134 of the needle tip 102 can be seen to extend radially outwardly from body axis 110 further than the remaining portion of the tip 102. A sloping surface 136 connects to an opening 140 of the aspiration passage that is coextensive with a lower surface 141 of the aspiration passage. The sloping surface 136 extends radially outward in the direction moving toward the open end of the tip 102 in a substantially straight surface defined by angle alpha. Angle alpha is the angle of sloping surface 136 with respect to the body axis 110. Angle alpha is less than 90 degrees and may be between 12 degrees to 90 degrees. Preferably the slope of surface 136, or angle alpha, of is less than or equal to 45 degrees. The sloping surface 136 further connects to a second interior surface 137 at a point 138, with second interior surface 137 being generally parallel to body axis 110. Dimension “A” is the length, along the body axis 110, of the second interior surface 137. Dimension “B” is the length component, along the body axis 110, of the sloping surface 136, while dimension “C” is the height component of the sloping surface 136. In the preferred embodiment, dimension “A” is greater than that of dimension “B”. Dimension “D” is the total height of the aspiration passage 124 at the needle distal end 99. In the first illustrated preferred embodiment of the needle 100, the tip 102 height “C” of the sloping surface 136 is at least one half of the total aspiration passage height “D”.

The sloping surface 136 is preferably manufactured in a secondary step of milling the needle tip 102. However, the sloping surface 136 may be created by other common manufacturing methods, such as being integrally formed in the needle body, or removed by etching, electrical discharge machining, or other material removal operations.

The inventor has found that a phacoemulsification needle with a flaring, off-center tip, such as the tip 102, provides an ideal hold on the nucleus of the eye during phacoemulsification. It is believed that the wide mouth of the flaring tip 102 having a large surface area, which is followed by a relatively narrower surface area aspiration passage 124, contributes to this advantageous feature. Further, it has been found that this configuration of the needle 100 improves fluid management in the eye to minimize flattening of the chamber of the eye. It is believed that the wide mouth of the flaring tip 102 having a large surface area, which is followed by a relatively narrower surface area aspiration passage 124, also reduces the relative amount of irrigation fluid aspirated during phacoemulsification as compared to prior art needles.

With reference to FIG. 5, a fragmentary portion or slice of the needle body 104 that lies proximally of the tip 102 (i.e., located toward the proximal end 106 of the needle 100 along the body central axis 110) is shown. The body 104 has an internal surface 200 from which a plurality of protrusions 210 extend inwardly toward the center of the needle body 104. The protrusions 210 are arranged in a spiraled or rifled pattern extending along the length of the needle body 104 along the central body axis 110. It is believed the rifled pattern of the protrusions 210 may assist in breaking up, dispersing, or otherwise degrading the aspirated portions of the nucleus that enter the aspiration passageway 124 to prevent or at least minimize clogging of the narrow passageway 124. The protrusions 210 preferably extend the full length of the aspiration passageway 124 from the tip 102 (FIG. 1) to the proximal end 106 of the needle 100 (FIG. 1). However, it will be understood that the protrusions 210 may extend only part way along the length of the needle body 104. The protrusions 210 in FIG. 5 have a generally semi-circular or arcuate shape when viewed in a cross-sectional plane that is normal to the central body axis 110 (FIG. 1). It will be appreciated that the protrusions 210 may have other cross-sectional shapes, such as square, triangular, or other polygonal or irregular shape improve the improve the aspiration properties.

The inventor has found that providing protrusions 210 within the aspiration passage 124 of a phacoemulsification needle with a flaring, off-center tip, such as the tip 102, may be especially advantageous in improving the fluid management in the eye during phacoemulsification to minimize flattening of the chamber of the eye, iris flutter, and/or clogging of the aspiration passage 124 due to blockages.

With reference to FIG. 6, an alternative embodiment of the internal features of the needle body 104 of the needle 100 are illustrated, wherein the alternative embodiment of the needle body is designated with the numeral 104A. It will be understood that FIG. 6 shows only a portion of the needle body 104A that lies proximally of the tip 102 (FIG. 1). The body 104A has an internal surface 200A in which a helical groove 210A extends between the proximal and distal ends of the needle body 104A. The groove 210A may advantageously assist in breaking up, dispersing, or otherwise degrading the aspirated portions of the nucleus that enter the aspiration passageway 124 (FIG. 4) to prevent or at least minimize clogging of the narrow passageway 124. The groove 210A preferably extends the full length of the aspiration passageway 124 from the tip 102 (FIG. 1) to the proximal end 106 of the needle 100 (FIG. 1). However, it will be understood that the groove 210A may extend only part way along the length of the needle body 104A.

The groove 210A in FIG. 6 has a generally screw-thread shape when viewed in a cross-sectional plane normal to the central axis 110 (FIG. 1). It will be appreciated that the groove 210A may have other cross-sectional shapes, such as square, circular, or other polygonal or irregular shape depending on the application. Furthermore, the pitch, depth, and/or angle of the groove 210A may be varied from what is illustrated. It will also be understood that the number of grooves 210A may be increased. It is believed that the groove 210A behaves in a similar manner as described above with respect to the protrusions 210 to minimize flattening of the chamber of the eye, iris flutter, and/or clogging of the aspiration passage 124 due to blockages thereof during the phacoemulsification of the nucleus of the eye. Preferably, the groove 210A is formed from a secondary machining process such as by drilling or cutting the needle body 104A.

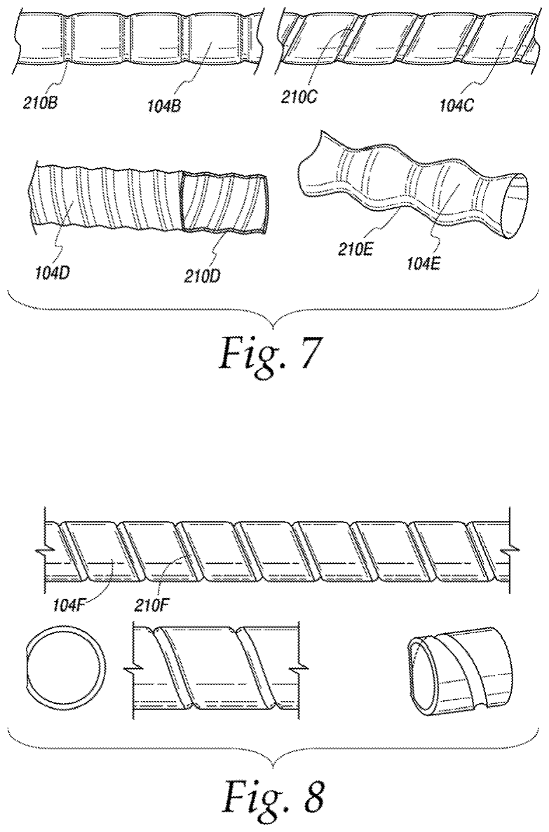

With reference to FIG. 7, four alternative embodiments of the needle body 104 of the needle 100 are illustrated, wherein the alternative embodiments of the needle body are designated with the numerals 104B, 104C, 104D, and 104E, respectively. It will be understood that FIG. 7 shows only portions of the needle body 104B, 104C, 104D, and 104E, which portions lie proximally of the tip 102 (FIG. 4).

Still referring to FIG. 7, the body 104B has a series of circumferential recessed portions or indented portions 210B when viewed externally, which results in circumferential projections or a rippled internal surface of the needle body 104B which defines the aspiration passage 124. Unlike the embodiments of the body 104 or 104A, which have substantially smooth cylindrical outer surfaces, the outer surface of the needle body 104B is noticeably indented with periodic indented portions 210B. It will be understood that in some alternative forms, not illustrated, the spacing of the indented portions 210B need not be regular or periodic, and instead may be irregularly spaced.

Still referring to FIG. 7, the body 104C has a series of angled recessed portions or indented portions 210C, which result in angled projections on the internal surface or an angled, rippled internal surface of the needle body 104C. It can be seen that the outer surface of the needle body 104C is noticeably indented with periodic indented portions 210C.

Still referring to FIG. 7, the body 104D has a single angled recessed portion or indented portion 210D arranged in a helical fashion about the body 104D which results in a single helical projection or ripple extending along the length of the internal surface of the needle body 104C.

Again, referring to FIG. 7 the body 104E has a series of sinusoidal recessed portions or indented portions 210E when viewed externally, which results in a sinusoidal rippled internal surface of the needle body 104E.

It will be understood that the indented portions 210B, 210C. 210D, and 210E, with their resulting internal projections, may assist in breaking up, dispersing, or otherwise degrading the aspirated portions of the nucleus that enter the aspiration passageway 124 (FIG. 4) to prevent or at least minimize clogging of the narrow passageway 124. The series of indented portions 210B, 210C, 210D, and 210E preferably extend in a periodic manner along the full length of the aspiration passageway 124 from the tip 102 (FIG. 4) to the proximal end 106 of the needle 100 (FIG. 1).

However, it will be understood that the series of indented portions 210B, 210C, 210D, and 210E may extend only part way along the length of the needle body 104B, 104C, 104D, and 104E. The pitch, depth, angle, number and shape of the indented portions 210B, 210C, 210D, and 210E may be varied from what is illustrated. Preferably, the indented portions 210B, 210C, 210D, and 210E are formed together with the remainder of the needle 100, without the need for a secondary machining process such crimping of the needle 100 to form the indented portions 210B, 210C, 210D, and 210E which may reduce manufacturing costs.

With reference now to FIG. 8, another alternative embodiment of the needle body 104 of the needle 100 is illustrated, wherein the alternative embodiment of the needle body is designated with the numeral 104F. It will be understood that FIG. 8 shows only portions of the needle body 104F, which lie proximally of the tip 102 (FIG. 4). The body 104F has a single angled recessed portion or indented portion 210F with a semi-circular shape arranged in a helical fashion about the body 104F.

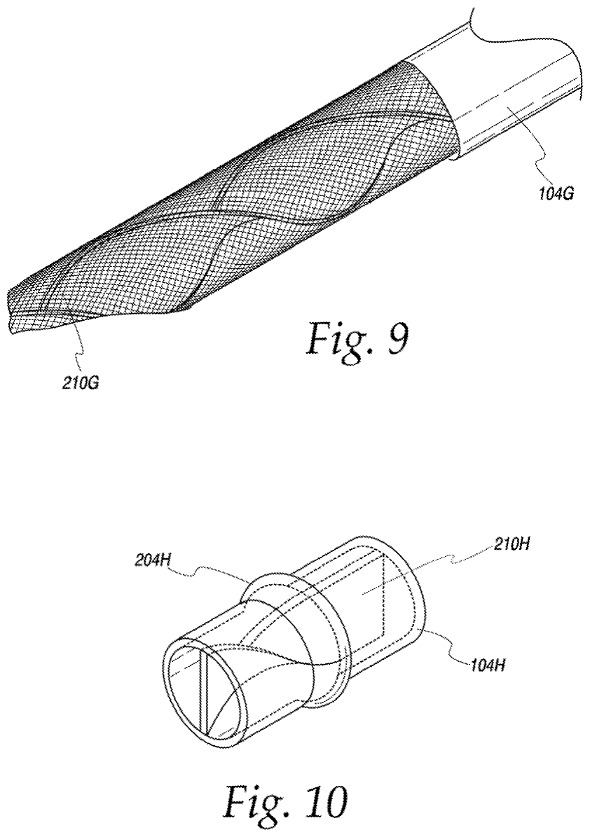

With reference now to FIG. 9, another alternative embodiment of the needle body 104 of the needle 100 is illustrated, wherein the alternative embodiment of the needle body is designated with the numeral 104G. It will be understood that FIG. 9 shows only a portion of the needle body 104G, which lies proximally of the tip 102 (FIG. 1). The body 104G has an internal twisted body or insert 210G which contacts aspirated tissues that travel through the hollow body 104G to assist in breaking up, dispersing, or otherwise degrading the aspirated portions of the nucleus that enter the aspiration passageway 124 (FIG. 4) to prevent or at least minimize clogging of the narrow passageway 124.

The twisted insert 210G preferably extends the full length of the aspiration passageway 124 from the tip 102 (FIG. 4) to the proximal end 106 of the needle 100 (FIG. 1). However, it will be understood that the insert 210G may extend only part way along the length of the needle body 104G. The period and shape of the twist may be varied from what is illustrated. Preferably, the insert 210G is either formed together with the remainder of the needle 100, without the need for a secondary machining process, or it is inserted in a secondary process by way of welding or a friction fit with the remainder of the needle 100.

With reference now to FIG. 10, yet another alternative embodiment of the needle body 104 of the needle 100 is illustrated, wherein the alternative embodiment of the needle body is designated with the numeral 104H. It will be understood that FIG. 10 shows only a portion of the needle body 104H, which lies proximally of the tip 102 (FIG. 1). The body 104H includes a raised circumferential portion 204H and a twisted body or insert 210H, both of which contact aspirated nucleus portions that travel through the hollow body 104H to assist in breaking up, dispersing, or otherwise degrading the aspirated portions of the nucleus that enter the aspiration passageway 124 (FIG. 4) to prevent or at least minimize clogging of the narrow passageway 124. The raised circumferential portion 204H has a corresponding circumferential internal recess, which allows for varying spacing between the internal surface of the body 104H and the insert 210H.

The twisted insert 210H preferably extends the full length of the aspiration passageway 124 from the tip 102 (FIG. 4) to the proximal end 106 of the needle 100 (FIG. 1). However, it will be understood that the insert 210H may extend only part way along the length of the needle body 104H. The period and shape of the twist may be varied from what is illustrated. Preferably, the insert 210H is either formed together with the remainder of the needle 100, without the need for a secondary machining process, or it is inserted in a secondary process by way of welding or a friction fit with the remainder of the needle 100. Preferably, the body 104H includes multiple raised circumferential portions 204H along its length.

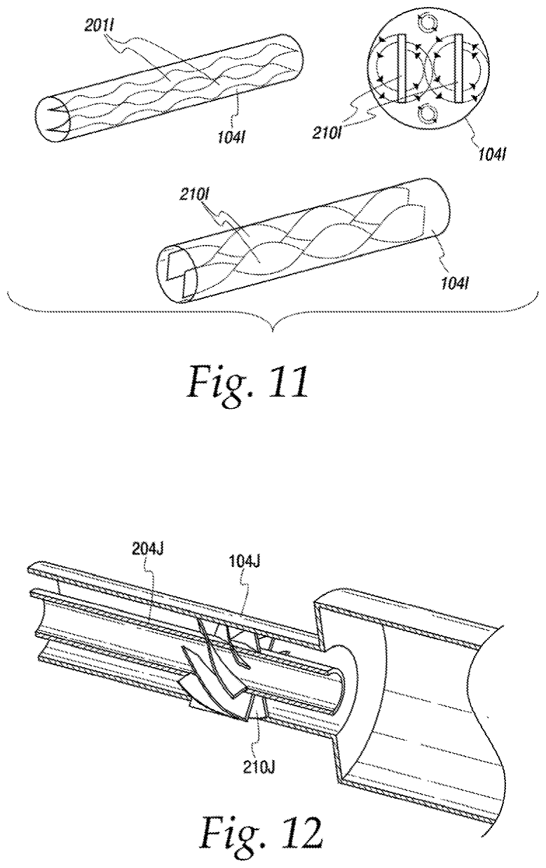

FIG. 11 illustrates another alternative embodiment of the needle body 104 of the needle 100, wherein the alternative embodiment of the needle body is designated with the numeral 104I. It will be understood that FIG. 11 shows only a portion of the needle body 104I, which lies proximally of the tip 102 (FIG. 1). The body 104I includes a pair of twisted bodies or inserts 210I, both of which contact aspirated nucleus portions that travel through the hollow body 104I to assist in breaking up, dispersing, or otherwise degrading the aspirated portions of the nucleus that enter the aspiration passageway 124 (FIG. 4) to prevent or at least minimize clogging of the relatively narrow passageway 124. The pair of inserts 104I allow for turbulent flow of material through the body 104I.

The twisted inserts 210I preferably extend the full length of the aspiration passageway 124 from the tip 102 (FIG. 4) to the proximal end 106 of the needle 100 (FIG. 1). However, it will be understood that one or both inserts 210I may extend only part way along the length of the needle body 104I. The period and shape of the twist may be varied from what is illustrated. Preferably, the inserts 210I are either formed together with the remainder of the needle 100, without the need for a secondary machining process, or they are inserted in a secondary process by way of welding or a friction fit with the remainder of the needle 100.

FIG. 12 illustrates another alternative embodiment of the needle body 104 of the needle 100, wherein the alternative embodiment of the needle body is designated with the numeral 104J. It will be understood that FIG. 12 shows only a portion of the needle body 104J, which lies proximally of the tip 102 (FIG. 1). The body 104J includes a central shaft or post 204J with a plurality of fixed fins or blades 210J extending radially therefrom which are exposed to the aspiration passage 124 (FIG. 4). The blades 210J contact the aspirated nucleus portions that travel through the hollow body 104J to assist in breaking up, dispersing, or otherwise degrading the aspirated portions of the nucleus that enter the aspiration passageway 124 to prevent or at least minimize clogging of the relatively narrow passageway 124.

Multiple sets of blades 210J may extend the full length of the aspiration passageway 124 from the tip 102 (FIG. 4) to the proximal end 106 of the needle 100 (FIG. 1). However, it will be understood that the blades 210J may extend only part way along the length of the needle body 104J. The number, angle, and shape of the blades 210J may be varied from what is illustrated. Preferably, the blades 210J are either formed together with the remainder of the needle 100, without the need for a secondary machining process, or they are inserted in a secondary process by way of welding or a friction fit with the remainder of the needle 100.

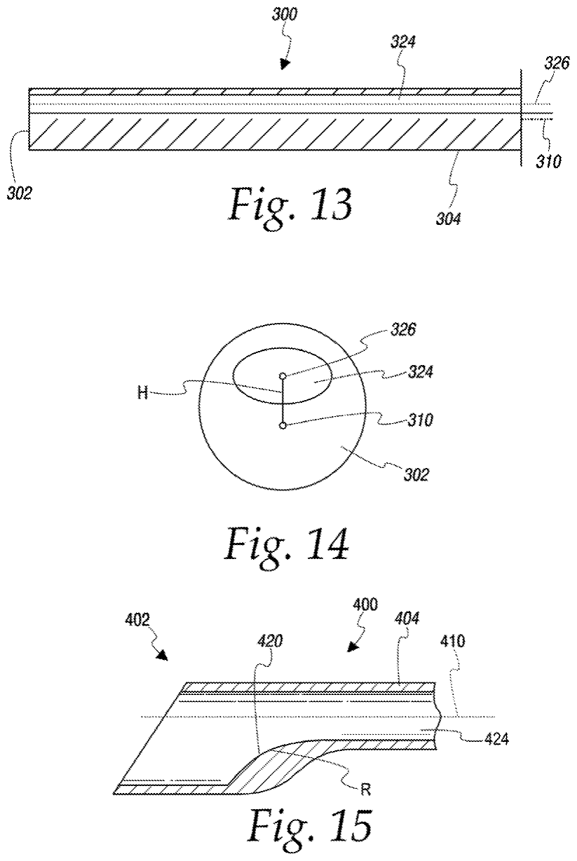

It will be understood that all of the above-described embodiments of the needle body 104, 104A, 104B, 104C, 104D, 104E, 104F, 104G, 104H, 104I, and 104J could be used on a needle without a flaring or offset tip. For example, any of the bodies 104, 104A, 104B, 104C, 104D, 104E, 104F, 104G, 104H, 104I, or 104J may be formed on a needle without a flaring tip such as that shown in FIGS. 13 and 14, designated as 300. As with the first illustrated embodiment of the needle 100, the needle 300 includes a tip 302, a central body 304 with a body axis 310, an aspiration passage 324 with its own central axis 326. The axes 310 and 326 are offset by a distance H (FIG. 14).

It will also be understood that all of the above-described embodiments of the needle body 104, 104A, 104B, 104C, 104D, 104E, 104F, 104G, 104H, 104I, and 104J could be used on a needle such as the needle 400 illustrated in FIG. 15, which has a flaring or offset tip 402 with different internal surfaces leading to the aspiration passage to prevent clogging. As with the first illustrated embodiment of the needle 100, the needle 400 includes a tip portion 402, and an elongate shaft or body 404 with a body central axis 410, and an aspiration passage 424 extending through the body 404. The tip 402 includes a curved, convex internal surface 420 leading from the relatively wider surface area of the mouth of the needle 400 toward the relatively narrower surface area of the aspiration passage 424, taken in a plane that is normal to the central body axis 410. The curved sloping surface 420 is defined by a radius “R” and forms a conoid shape in three dimensions, while forming a convex curve in two dimensions. The radius “R” of the sloping surface 420 is preferably between 0.35 to 0.9 mm. This conoid shape of the sloping surface 420 may reduce the amount of removed tissue material that is deflected from the sloping surface 420 and thus improving the efficiency of the aspiration of the needle 400 having a flaring tip 402.

The inventive needle bodies 104, 104A, 104B, 104C, 104D, 104E, 104F, 104G, 104H, 104I, and 104J, which have a blockage reduction means as described above, may advantageously be used with a variety of vibratory handpieces which can impart a longitudinal, torsional, elliptical, and/or blended vibrations to the needle. Furthermore, such an improved needle may eliminate the need for employing an expensive fluidics management system when performing phacoemulsification on tissues of the eye.

The inventive needle bodies 104, 104A, 104B, 104C, 104D, 104E, 104F, 104G, 104H, 104I, and 104J, which have a blockage reduction means as described above, may advantageously be used with a needle that is not generally straight, and that is bent, stepped, or angled along its length.

It should be understood that although the embodiments shown depict specific wall configurations of the needle, the invention should not be so limited. Selected walls or wall portions of the phacoemulsification needle can be manufactured to various thicknesses.

The foregoing disclosure of specific embodiments is intended to be illustrative of the broad concepts comprehended by the invention.

1. A phacoemulsification needle for emulsifying body tissue, said needle adapted to be attached to a phacoemulsification handpiece for imparting a vibration to said needle, said needle comprising:
a hollow needle body having a distal end, a proximal end, and an internal surface defining an aspiration passage extending between said proximal and distal ends, said proximal end for mounting said needle body to a phacoemulsification handpiece,
said aspiration passage having a longitudinally-extending central body axis,
said needle body defining a tip at said distal end,
said needle body having a blockage reduction means located within said aspiration passage at a location proximal of said tip, wherein said blockage reduction means is one of: (i) a protrusion extending radially inward from said internal surface; (ii) a groove extending into said internal surface;
(iii) at least one insert within said aspiration passage; (iv) at least one blade located within said aspiration passage; or (v) at least one indented portion of said needle body.

2. The phacoemulsification needle of claim 1 wherein said aspiration passage is enlarged at said tip, said aspiration passage in said tip having a surface area that is greater than a surface area of said aspiration passage in said body, taken in a plane that is normal to said central body axis.

3. The phacoemulsification needle of claim 2 wherein said tip has a radially offset portion, said radially offset portion having an interior sloping surface that slopes radially inward in a direction along said body axis toward said body proximal end of said needle body.

4. The phacoemulsification needle as recited in claim 3 wherein said interior sloping surface is a convex curve.

5. The phacoemulsification needle as recited in claim 3 wherein said interior sloping surface is substantially straight.

6. The phacoemulsification needle as recited in claim 1 wherein at least a portion of said tip has a textured surface that is at least one of: (i) an exterior surface of said tip; or (ii) an interior surface of said tip.

7. The phacoemulsification needle as recited in claim 1 wherein said body has a length along said central body axis, said blockage reduction means extends along a majority of the length of said needle body.

8. The phacoemulsification needle as recited in claim 1 wherein said body has a length along said central body axis, said blockage reduction means extends a distance of less than half of the length of said needle body.

9. The phacoemulsification needle as recited in claim 1 wherein said blockage reduction means is a plurality of protrusions extending radially inward from said internal surface, said plurality of protrusions arranged in a rifled pattern on said internal surface.

10. The phacoemulsification needle as recited in claim 1 wherein said blockage reduction means is at least one a helical groove extending within said internal surface.

11. The phacoemulsification needle as recited in claim 1 wherein said blockage reduction means is a plurality of periodic, indented portions of said needle body.

12. The phacoemulsification needle as recited in claim 11 wherein said blockage reduction means is a plurality of periodic, indented portions of said needle body, said indented portions having a sinusoidal shape when viewed in cross section in a plane that contains said central body axis.

13. The phacoemulsification needle as recited in claim 11 wherein said blockage reduction means is a plurality of periodic, circumferential indented portions of said needle body.

14. The phacoemulsification needle as recited in claim 11 wherein said blockage reduction means is a plurality of periodic, indented portions of said needle body that are angled with respect to a plane that is normal to said central body axis.

15. The phacoemulsification needle as recited in claim 1 wherein said blockage reduction means is at least one twisted insert extending within said aspiration passage.

16. The phacoemulsification needle as recited in claim 15 wherein said blockage reduction means is a pair of twisted inserts extending within said aspiration passage.

17. The phacoemulsification needle as recited in claim 1 wherein said blockage reduction means is a plurality of radially extending blades extending within said aspiration passage.

18. A method of performing phacoemulsification, the method comprising the steps of:
obtaining the phacoemulsification needle of claim 1;
assembling said proximal end of said phacoemulsification needle with a vibratory handpiece;
imparting a vibration to said needle with said handpiece to emulsify tissues in the eye; and
aspirating emulsified tissues into said aspiration passageway to contact said blockage reduction means.
